(19) United States
(12) Patent Application Publication
(10) Pub. No.: US 2022/0313427 A1
(43) Pub. Date: Oct. 6, 2022

(54) System To Facilitate Tether Re-Tensioning

(71) Applicant: Tendyne Holdings, Inc., St. Paul, MN (US)

(72) Inventor: Zachary R. Vidlund, Robbinsdale, MN (US)

(73) Assignee: Tendyne Holdings, Inc., St. Paul, MN (US)

(21) Appl. No.: 17/691,307

(22) Filed: Mar. 10, 2022

Related U.S. Application Data
(60) Provisional application No. 63/170,772, filed on Apr. 5, 2021.

(51) Int. Cl. A61F 2/24

(52) U.S. Cl. CPC A61F 2/2418; A61F 2220/0008; A61F 2220/0075; A61F 2/86

(57) ABSTRACT

A system comprising a prosthetic heart valve having a frame, prosthetic leaflets within the frame configured to allow blood to flow through the prosthetic heart valve in an antegrade direction but to substantially block blood from flowing through the prosthetic heart valve in a retrograde direction, an anchor configured to be secured against a ventricular apex of a heart of a patient, a tether coupled to the frame and through the anchor, and an end of the tether configured to extend beyond the anchor when the tether is fixed to the anchor, and a tether adjustment member having a collapsible tube defining an open leading end configured to receive the second end of the tether, and a wire leader extending from a second trailing end of the tube.

### CROSS-REFERENCE TO RELATED APPLICATIONS

This application claims priority to U.S. Provisional Patent Application No. 63/170,772, filed Apr. 5, 2021, the disclosure of which is hereby incorporated by reference herein.

### BACKGROUND OF THE DISCLOSURE

The present disclosure relates to devices and methods for securing, tensioning, and/or re-tensioning a tether of a prosthetic heart valve.

Various options are available to maintain a prosthetic heart valve in a desired position within a native heart valve annulus of a patient. For example, the position of a surgical prosthetic heart valve may be maintained by suturing the prosthetic heart valve into the patient's native heart valve annulus. Collapsible and expandable prosthetic heart valves, on the other hand, may be maintained in a desired position by exerting radial forces against the native heart valve annulus and/or surrounding tissue. It may additionally be beneficial to assist collapsible and expandable prosthetic heart valves in maintaining the desired position through the use of a tether that extends from the prosthetic heart valve to an anchor on an exterior portion of the patient's heart. However, during or after the surgery, the tension on the tether and the position of the prosthetic heart valve may need to be adjusted, for example, if the prosthetic heart valve deviates from its intended position, or if the tension on the tether changes for any reason. In current systems, it may be difficult to adjust tension on the tether after the tension has been set.

Thus, it would be preferable for a tether and the corresponding anchor to allow for post-implantation modifications to adjust the tension of the tether.

### BRIEF SUMMARY

One aspect of the disclosure provides for a system that comprises a prosthetic heart valve having a frame, prosthetic leaflets within the frame configured to allow blood to flow through the prosthetic heart valve in an antegrade direction but to substantially block blood from flowing through the prosthetic heart valve in a retrograde direction, an anchor configured to be secured against a ventricular apex of a heart of a patient, a tether having a first end, a second end, and an intermediate portion between the first end and the second end, the first end of the tether coupled to the frame, the intermediate portion configured to be received within the anchor, and the second end configured to extend beyond the anchor when the tether is fixed to the anchor, and a tether adjustment member having a collapsible tube defining an open leading end configured to receive the second end of the tether, and a wire leader extending from a second trailing end of the collapsible tube.

Another aspect of the disclosure provides for a method of adjusting tension on a tether that is coupled to a prosthetic heart valve implanted within a heart of a patient that comprises accessing the tether, the tether having a first end coupled to the prosthetic heart valve, a second end extending from the anchor, and an intermediate portion received within the anchor, the intermediate portion being between the first end and the second end, after accessing the tether, engaging the second end of the tether with a collapsible tube so that the second end of the tether is received within a first leading end of the collapsible tube, securing the collapsible tube to the tether, and tensioning the tether by applying a proximally directed force to the collapsible tube.

### BRIEF DESCRIPTION OF THE DRAWINGS

These and other features, aspects, and advantages of the present disclosure will become better understood with regard to the following description, and accompanying drawings.

FIG. 1 depicts a cut-away view of a portion of a heart with a prosthetic mitral valve implanted therein and an epicardial anchor device anchoring the mitral valve in position.

FIGS. 2-5 depicts a braid used to tension an excess tether in accordance with an aspect of the disclosure.

FIG. 6 depicts a braid in use with a tool in accordance with another aspect of the disclosure.

FIG. 7 depicts a braid with teeth in accordance with another aspect of the disclosure.

FIG. 8 depicts a braid in use with a leader having a braided section and a solid section in accordance with another aspect of the disclosure.

### DETAILED DESCRIPTION

Some devices for anchoring a medical device, such as a collapsible and expandable prosthetic heart valve, can include securing one or more tethers extending from the medical device to an anchor positioned on the heart, such as along an exterior portion of the ventricular wall. In one exemplary valve replacement procedure, the prosthetic heart valve may be delivered to a native valve annulus while in a collapsed state, and then allowed to expand to at least partially secure the prosthetic heart valve within the native valve annulus. If the prosthetic heart valve is for use in replacing a native mitral valve, a flared inflow end of the prosthetic heart valve may help prevent migration of the prosthetic heart valve into the left ventricle, while a tether attached to the prosthetic heart valve may assist in preventing migration of the prosthetic heart valve into the left atrium. During implantation, while the prosthetic heart valve is positioned within the valve annulus, a first end of the tether may be coupled to the prosthetic heart valve, and a second end of the tether may exit the heart, for example via a puncture in the left ventricular apex. While the second end of the tether is positioned outside the heart, an anchor may be slid over the tether until the anchor sits along the exterior portion of the ventricular wall such that an excess portion of the tether extends past the anchor outside the heart. For example, the anchor may include a central aperture that allows the anchor to slide over the free end of the tether until the anchor is in contact with the exterior tissue of the heart. The tether may then be tensioned until the prosthetic heart valve is at a desired tension and position within the patient's heart valve annulus. Once that desired tension is reached, the tether may be fixed to the anchor and the excess portion of the tether may be cut off and removed, thereby preventing excess length of the tether from freely floating within the patient's body and potentially interfering with the patient's surrounding anatomy. In one example, a portion of the anchor may be rotated to drive a pin that resides within the anchor through the tether to fix the tether to the anchor at the desired tension. In such procedures, it should be understood that much or most of the tether may be formed from a fabric (e.g. a braided polymer). A proximal end of the tether (or the end closest to the surgeon) may be coupled to a thin wire, such as a leader wire formed of nitinol. The leader wire may facilitate guiding devices, such as the anchor, over the tether. The leader wire may be helpful because it may be more difficult to guide devices over the tether if the terminal free end of the tether were formed of braided fabric.

However, during removal of the excess portion of the tether, the leader wire (if included) is typically removed from the tether. After the excess tether length is removed, there may be a desire to re-tension the tether. For example, such additional tensioning may be desirable if the prosthetic heart valve is not implanted in a desired position during surgery, or if the position shifts after surgery. Alternatively, tension readjustment may be desired after the surgery is complete and an interval of time has lapsed. In such an instance, the prosthetic heart valve may have been repositioned through wear or unintentional position shifts, or the native heart anatomy may have adjusted. For example, the tension on the tether may cause the ventricular wall to change shape, which in turn may reduce the tension of the tether. Thus, it may be beneficial to re-capture an excess tether length to allow for further tightening. However, as noted above, after the excess length of tether is removed, there is typically only a small length of tether that protrudes beyond the anchor (e.g. about 3 cm-10 cm, including about 5 cm), and the terminal end of that tether is typically formed of braided fabric, which may be difficult to manipulate without the benefit of the leader wire that was previously removed.

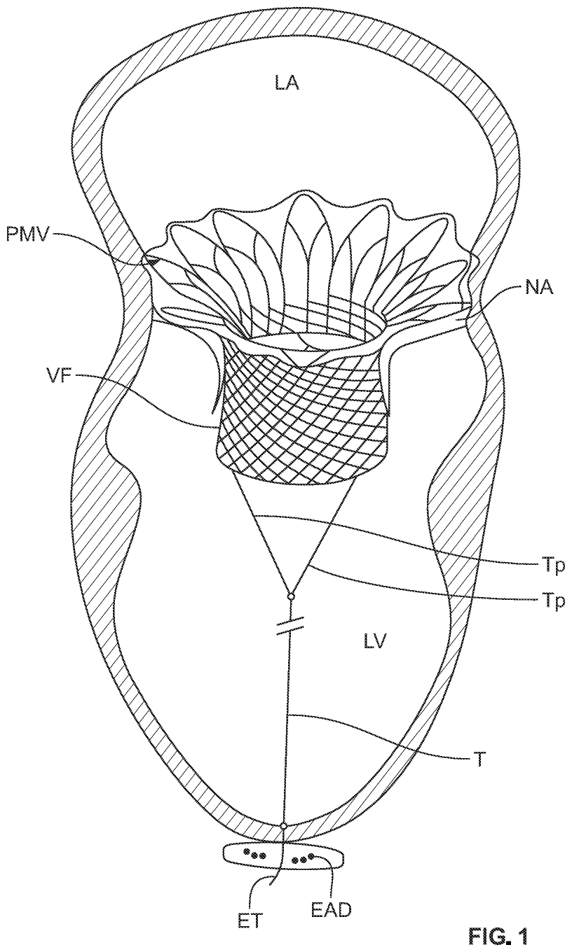

FIG. 1 is a cross-sectional illustration of the left ventricle LV and left atrium LA of a heart having a transcatheter prosthetic mitral valve PMV deployed therein and an epicardial anchor device EAD as described herein securing the prosthetic mitral valve in place. FIG. 1 illustrates the prosthetic mitral valve PMV seated into the native annulus NA of the valve and held there using a valve frame VF of the prosthetic mitral valve, the radial tension from the native leaflets, and a ventricular tether T secured with attachment portions Tp to the prosthetic mitral valve and to the epicardial anchor EAD. A small portion of tether T extends past epicardial anchor EAD, leaving a bit of excess tether ET outside of the heart. It should be understood that this excess tether ET may be generally representative of the length of tether T remaining after the remainder of the tether T is cut away. An atrial flare portion (not separately labeled) of the valve frame VF may be positioned in the left atrium LA of the heart to prevent migration of the prosthetic mitral valve PMV into the left ventricle LV. Various embodiments of an epicardial anchor device are described in more detail below with reference to specific embodiments. Additional features of epicardial anchor devices are also described in U.S. Patent Publication No. 2016/0143736, the disclosure of which is hereby incorporated by reference herein.

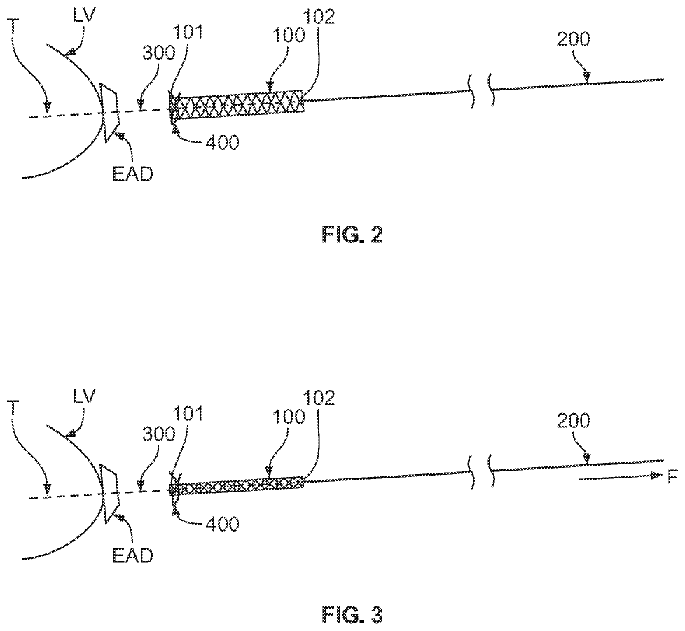

FIG. 2 depicts an exemplary braid 100 in a relaxed condition (i.e., in the absence of applied forces) coupled with a leader 200, with the braid 100 surrounding and being coupled to a length of excess tether 300. It should be understood that FIG. 2 illustrates a scenario in which the prosthetic mitral valve PMV has been implanted, the tether T has been tensioned and locked to the epicardial anchor device EAD, and the tether T has been cut so that only a small length of excess tether 300 extends beyond the epic ardial anchor device EAD. If it becomes desirable to adjust the tension of tether T, the braid 100 may be used as described below to facilitate such adjustment. In the illustrated configuration, braid 100 is substantially tube shaped with a first untensioned diameter along its length, including at a leading or first end 101, and a trailing or second end 102. Braid 100 may have a woven structure made of a number of wires, for example braided nitinol or other biocompatible and/or shape memory metals or metal alloys. This woven structure facilitates braid 100 to be radially compressed when the braid is axially tensioned and stretched, thus decreasing the untensioned diameter of braid 100 along a portion of the braid and providing a compressive force along that portion of the braid. Leader 200 may be a metal guidewire that is secured to end 102 through suturing, welding, glue, or the like. Further, it should be understood that although the collapsible tube 100 is generally referred to herein as a braid, other structures may be suitable. For example, any collapsible tube may be able to perform a generally similar function, whether that tube is braided, laser-cut from a tube to form a collapsible and expandable stent, or any other form of collapsible tube.

Excess tether 300 can be a portion of excess tether (e.g. between 3-10 cm length) extending from an epicardial anchor EAD after a prosthetic mitral valve PMV (not shown) is securely tensioned with epicardial anchor EAD through a tether T. A terminal free end of excess tether 300 is accessed, for example by creating an incision in the patient's chest if the prior implantation procedure has been fully completed. That terminal free end of excess tether 300 may be received through leading end 101 of braid 100 so that a portion of excess tether 300 is received within and/or surrounded by the braid while the braid is in the relaxed or unbiased condition. While in this initial relaxed condition, excess tether 300 can be secured to braid 100 through, for example, a securement mechanism 400 at or near leading end 101. Securement mechanism can be 400 can be a suture knot, weld, glue, or the like.

While in the relaxed condition, the untensioned diameter of leading end 101 of braid 100 is larger than a diameter of excess tether 300. This difference in diameter size can allow for braid 100 to more easily receive excess tether 300, as discussed further below.

FIG. 3 depicts braid 100 in a tensioned condition being pulled axially with a proximally directed pulling force F. As force F tensions braid 100, the diameter of the braid decreases so that the braid has a tensioned diameter smaller than the untensioned diameter. The braid 100 is prevented from sliding relative to the excess tether 300 via the securement mechanism 400. In this manner, braid 100 can apply a radially compressive force to excess tether 300.

Braid 100 and leader 200 can be made of stainless steel, nitinol, or other biocompatible material, but it should be understood that braid 100 and leader 200 need not be formed of the same material. Excess tether 300 can be made of a braided or woven fabric as noted above. Braid 100 can have a varying amount of pick density (e.g., picks per inch) so that the density of wire strands can change along a given length of the braid. For example, the pick density can be increased near leading and trailing ends 101, 102 to minimize the chance that loose ends of braid 100 extend from the braid. Alternatively, braid 100 can have a constant pick density along its length.

The wires may be round or flat. In one example, the wires may be flat to provide an increased amount of surface area to contact excess tether 300 with while reducing the overall diameter of braid 100. It should be understood that as the braid 100 transitions from the untensioned condition to the tensioned condition over excess tether 300, significant friction forces may be created between the braid 100 and the excess tether 300, due to the compressive force of the braid 100 and surface area of contact between the braid 100 and the excess tether 300. Thus, it may be desirable for the wire strands of braid 100 to include flat faces that contact excess tether 300 to increase such surface area of contact, but such a construction is by no means necessary to achieve the desired forces to lock the braid 100 onto the excess tether 300.

FIGS. 2-5 depict braid 100 in use. FIG. 4 depicts a tether T and epicardial anchor device EAD with excess tether 300 extending from epicardial anchor device EAD. Once it is determined that tether T requires re-tensioning, another procedure can be performed to insert braid 100 into a patient's anatomy to facilitate such re-tensioning. In some circumstances, the re-tensioning may be desired very shortly (e.g. immediately) after the original tether is cut, and thus re-entry into the patient may not be necessary. In the initial, relaxed condition, leading end 101 of braid 100 has a larger diameter than the end of excess tether 300.

Braid 100 is then maneuvered and aimed by an instrument (not shown), such as forceps, pliers or the like, gripping the braid or manually by a surgeon's hand to receive an end of the excess tether 300 within the braid, as shown in FIG. 5. Once an end of excess tether 300 is received through leading end 101 and a portion of excess tether 300 is received within a portion of braid 100, excess tether 300 and braid 100 are secured together, preferably near leading end 101, as shown in FIG. 2. For example, a securement mechanism 400 can secure excess tether 300 to braid 100 by knotting, welding, or gluing excess tether 300 to securement mechanism 400.

Braid 100 is then tensioned by feeding leader 200 through a tensioning instrument and the tensioning instrument pulling the leader in a proximal direction away from epicardial anchor device EAD and a tensioning force F is applied to braid 100 to axially tension the braid. However, it should be understood that this tensioning may be performed manually. In some uses, the initial tensioning (described below) may be performed manually, with further tensioning being performed with the assistance of a tensioning instrument. During an initial tensioning phase, securement mechanism 400 holds leading end 101 of braid 100 while the braid is stretched along an axial direction away from the epicardial anchor device EAD without further tensioning (or without significantly tensioning) excess tether 300. In this initial phase, a threshold amount of force is required to further tension excess tether 300 (and, by extension, tether T) while a lesser threshold of force is required to begin tensioning braid 100. In other words, an initial applied pulling force F axially stretches the braid without further tensioning excess tether 300. Braid 100 will decrease in diameter until a tensioned diameter of the braid is about equal to or smaller than a diameter of the portion of excess tether 300 received within the braid, applying a compressive force to excess tether 300. This initial applied pulling force F may be applied manually or with the assistance of a tensioning tool, and is primarily intended to create high friction forces between the braid 100 and the excess tether 300 in preparation for adjusting the tension of excess tether 300.

Once the braid 100 has decreased in diameter and “bites” or otherwise grips onto the excess tether 300, additional tensioning force F may be applied to tension excess tether 300, as depicted in FIG. 3. In this second tensioning phase, braid 100 will be sufficiently stretched so that the compressive and/or frictional forces applied between the braid and the excess tether 300 become the primary mechanism of securing the braid to the excess tether 300. Thus, securement mechanism 400 may not be required to withstand significant forces during the second phase of tensioning. The compressive and/or frictional force of braid 100 secures the portion of excess tether 300 received within the braid so that continued pulling of the braid will pull the excess tether away from epicardial anchor device EAD, once the epicardial anchor device is unpinned (or otherwise unlocked or uncoupled) from the excess tether.

As described above, epicardial anchor device EAD is engaged to tether T through a locking pin mechanism in the epicardial anchor device EAD, so, once excess tether 300 is sufficiently secured to the braid 100 as to be able to tension both the excess tether and tether T, a locking instrument (not shown) can receive leader 200 and the excess tether to engage epicardial anchor device EAD and unlock the pinning mechanism. In this manner, tether T is now free to be further tensioned by tensioning excess tether 300 through braid 100. Braid 100 can continue to be pulled until a desired tension of tether T is reached. Once that desired tension is achieved, the locking instrument can then lock epicardial anchor device EAD to tether T at this new tension by re-engaging the locking pin mechanism. After the tether T is locked to the EAD at the desired tension, the braid 100 may be decoupled from the excess tether 300, including, for example, by cutting the excess tether 300 just distal to securement mechanism 400.

In the embodiment described above, the braid 100 may have the untensioned, larger diameter when in a relaxed condition. In other words, the braid 100 can slip over the excess tether 300 when in the relaxed condition, and must be axially elongated to decrease the diameter of the braid 100 to properly grip the excess tether 300. However, in an alternative example, the braid 100 can have an inner diameter that is equal to, or smaller than, the outer diameter of excess tether 300 when the braid 100 is in the relaxed condition. With this configuration, braid 100 can be initially axially compressed so that leading end 101 and trailing end 102 are pushed towards each other to temporarily cause the inner diameter of braid 100 (and at least leading end 101) to be larger than in the relaxed condition. In this compressed condition, the inner diameter of leading end 101 can be larger than the outer diameter of excess tether 300, so that the braid 100 may be slipped over the excess tether 300 when temporarily axially collapsed.

In use, after braid 100 receives a portion of excess tether 300, the axial compression may be released to return to the braid to its relaxed condition over the portion of the excess tether. This configuration may allow for the braid 100 to grip the excess tether in the relaxed condition, which may eliminate the need for securing mechanism 400, although the securing mechanism 400 may be still be used to provide additional securement. It should be understood that the relaxed diameter of the braid 100 may be set by any suitable means, including for example heat setting shape-memory materials forming the strands of the braid 100.

In yet further examples, an inner diameter at leading end 101 can be different from the inner diameter of the rest of braid 100. For example, leading end 101 can have an inner diameter equal or smaller than an outer diameter of excess tether 300 while the rest of braid 100 can have an inner diameter greater than the outer diameter of the excess tether, thus only requiring the portion of the braid near leading end 101 to be expanded. When the inner diameter of the portion of braid 100 near leading end 101 is compressed, the friction applied by that portion would engage excess tether 300.

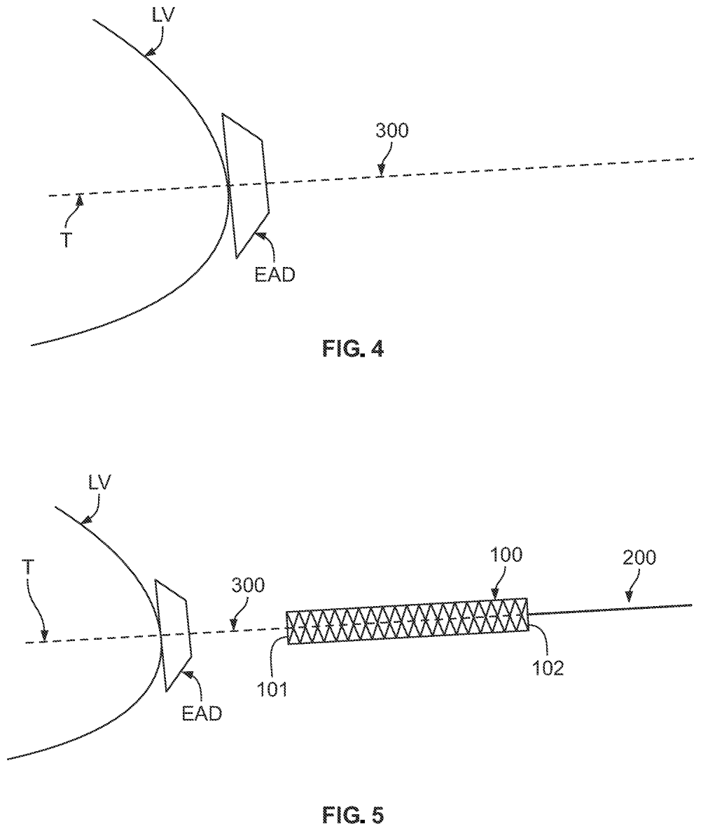

In a yet further alternative example, where the outer diameter of excess tether 300 is equivalent or larger than an inner diameter of braid 100 in a relaxed condition, a tool may be used to facilitate the insertion of the excess tether within the braid. FIG. 6 depicts braid 100 with a tool 500 attached to the braid at leading end 101. Tool 500 can be attached to braid through a glue, knot, or other means of temporary attachment. However, tool 500 need not be fixedly attached to the braid 100 at all. Tool 500 can be made of two or more parts, such as two halves as shown in FIG. 6. Tool 500 can be funnel shaped so that a substantially straight portion of tool 500 is received in braid 100 and a curved portion of the tool extends away from the braid facing epicardial anchor device EAD (not shown). In other words, when the two halves of tool 500 are assembled, the straight and narrow portion may have an outer diameter that is smaller than the inner diameter of braid 100 when in the relaxed condition. The curved portion of tool 500 defines a diameter larger than the diameter of excess tether 300, and larger than an inner diameter of braid 100 when in the relaxed condition. The tool 500 may be formed of two or more portions that can be assembled together and, when in the assembled condition, have a funnel shape. Preferably, tool 500 is formed as two similar or identical halves that can be assembled to form the funnel. The tool 500 may include features to allow for the assembly to maintain the assembled condition via a snap fit or other locking mechanism. However, in other embodiments, the tool 500 need not include any active coupling mechanisms to maintain the tool 500 in the assembled condition. In a further alternative, tool 500 can be one unitary piece made of a malleable material (e.g., plastic or the like) with one or more slits or perforations along its length.

In use, with this example, the two halves of tool 500 can be assembled to form the funnel shape, and the tool 500 can be attached to braid 100 by sliding the straight narrow portion of the funnel shape of tool 500 into the leading end 101 of the braid 100. This may be performed prior to insertion of the braid within a patient. Because the large funnel end of tool 500 has a diameter that is significantly larger than the inner diameter of braid 100 in the relaxed condition, the tool 500 may be pulled proximally, while a portion of the braid 100 is maintained stationary, to pull the leading end 101 of braid toward the trailing end 102 of the braid. The interaction between the large funnel end of the tool 500 and the leading end 101 of braid 100 results in the inner diameter of the braid near the leading end 101 increasing to more easily receive a portion of excess tether 300 into braid 100. It should be understood that when tool 500 is assembled, a passageway may extend through an interior of the assembled tool 500 that may receive the excess tether 300 therethrough. Thus, while the tool 500 is forcing the leading end 101 of the braid 100 into a large-diameter condition, the excess tether 300 may be passed into the tool 500 and into a portion of the braid 100, as shown in FIG. 6. Once a portion of excess tether 300 is received into braid 100, tool 500 can be detached from the braid. For example, one half of the tool 500 may be slipped out of the braid 100, and then the other half of the tool may be slipped out of the braid 100. Where tool 500 is a unitary piece with one or more slits or perforations along its length, removing the tool may include pulling portions of the tool adjacent the slit(s) or perforation(s) in a direction perpendicular to the slit(s) or perforation(s) to “peel” the tool away from braid 100 and excess tether 300. As the tool 500 is removed from the braid 100, the braid will begin to transition to its relaxed condition, clamping over (or biting into) the excess tether 300 in the process.

In some embodiments, after the portion of excess tether 300 is received into braid 100, and before or after tool 500 is detached from the braid, a securement mechanism 400 (not shown) can be used to additionally secure the excess tether to the braid. Although the above description of tool 500 is in regards to an embodiment of braid 100 where an outer diameter of excess tether 300 is equivalent or larger than an inner diameter of braid 100 in a relaxed condition, it is understood that the tool can be used in other embodiments where the inner diameter of the braid is larger than the outer diameter of the excess tether. In those embodiments, the primary purpose of the tool 500 may be to facilitate easy insertion of the excess tether 300 into the interior of the braid 100, because the funnel shape of the tool 500 (when assembled) may facilitate easy guidance of the excess tether 300 into the leading end 101 of the braid 100.

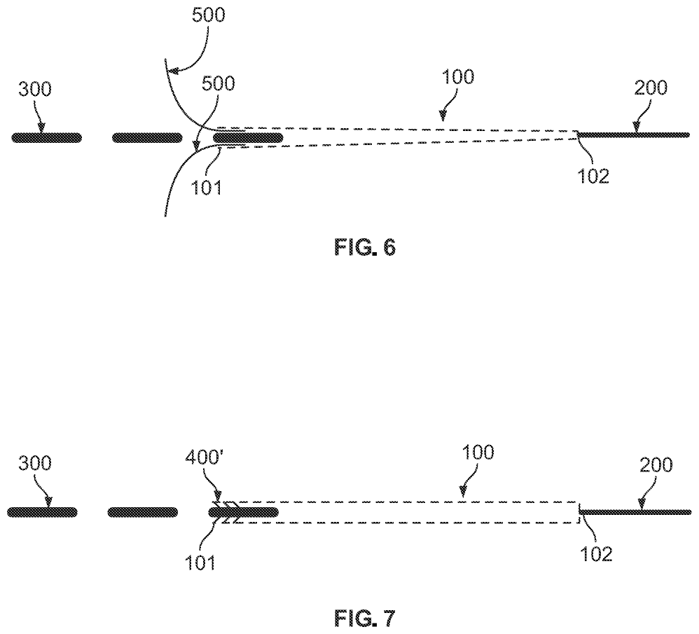

In yet another alternative example, FIG. 7 depicts excess tether 300 being secured to braid 100 through teeth 400′ extending radially inwardly and proximally along the braid in lieu of securement mechanism 400. Such teeth can be angled in a proximal direction away from the epicardial anchor device EAD so that a portion of excess tether 300 can be inserted in braid 100 through leading end 101 without engaging the tips of the teeth until braid 100 slides proximally away from the epicardial anchor device EAD relative to the portion of excess tether received within the braid. In effect, this provides the ability for the excess tether 300 to slide in one direction relative to braid 100, but not in the opposite direction. However, in other examples, a securement mechanism 400 can be used in addition to teeth 400′ to better secure excess tether 300 to braid 100. Although FIG. 7 depicts multiple teeth 400′ being used, other examples may include only using a single tooth.

In use, with this example, braid 100 can be maneuvered to receive a portion of excess tether 300 so that the portion of the excess tether can slide along teeth 400′ away from the epicardial anchor device EAD relative to the braid 100 without getting caught on the teeth. Immediately after receiving the portion of excess tether 300, braid 100 can be tensioned so that teeth 400′ can engage that portion of the excess tether, thus securing the braid to the excess tether and allowing for the braid to be further tensioned.

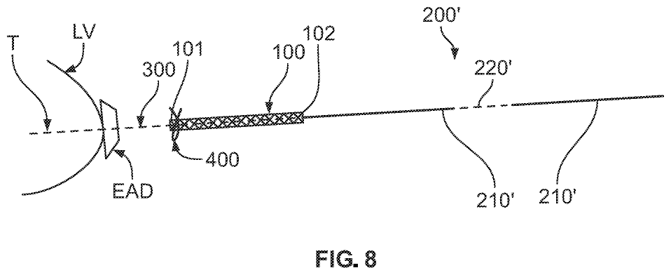

Although the above leader 200 is described as being a monolithic and/or uniform piece of metal wire, in other embodiments the leader can have a portion along its length that is structurally or materially different to facilitate additional re-tensioning options or engagement with an instrument such as a tensioning device. For example, FIG. 8 depicts braid 100 coupled to leader 200′ at or near the trailing end 102 of the braid. Leader 200′ includes solid sections 210′ and a braided section 220′. Solid sections 210′ can be a solid piece of metal wire as described for leader 200, above. Braided section 220′ can have a braided or woven structure that is the same or different material as solid sections 210′ (e.g., metal, fabric, or the like). In the particular illustrated example, braided section 220′ includes a braided or woven polymer or fabric structure that is similar to the structure of tether T. Braided section 220′ can be secured at both ends to solid sections 210′. It is understood that there can be any number of solid sections 210′ or braided sections 220′, such as two solid sections and two braided sections.

In use, once excess tether 300 is sufficiently secured to the braid 100 as to be able to tension both the excess tether and tether T, an instrument (e.g., a tensioning instrument, not shown), can engage leader 200′ by feeding the leader through the instrument. Some tensioning instruments may have a clamping-type feature in order to secure the leader 200′ to the tensioning instrument. Such a clamping-type feature may be best suited to clamp over a solid metal wire, such as solid sections 210′ of leader 200′. Other tensioning instruments may instead have a pinning-type feature, which may be similar to the pinning mechanism within the epicardial anchor device EAD. Such pinning mechanism may be best suited to be driven through a polymer and/or fabric braided structure, such as braided section 220′ of leader 200′. The tensioning instrument can then engage either braided section 220′ or solid section 210′ depending on the particular tensioning device. Once the tensioning device is secured to the leader 200′, the tensioning device may be used to further tension (or re-tension) excess tether 300. Although clamping-type and pinning-type coupling mechanisms are described above, other mechanisms may also be suitable. For example, in another method of engagement, the tensioning instrument can engage braided section 220′ by wrapping the braided section (and/or the solid section 210′) around a portion of the instrument (e.g., a spool or winding mechanism) to tension excess tether 300 by winding the leader 200′.

Although the above system is described for use with re-tensioning a prosthetic heart valve using the same epicardial anchor device, it is understood that the above method can be used to replace the epicardial anchor device with a new epicardial anchor device, whether or not tether tension adjustment is desired. For example, once the braid is secured to the excess tether through one of the methods described above, an instrument can unlock the old epicardial anchor device's engagement with the tether the old epicardial anchor device may be slid proximally over the braid and leader. Then, a new epicardial anchor device (not shown) can be slid distally over the leader and braid to be implanted in place of the old epicardial anchor device. This can be beneficial when the epicardial anchor device is damaging the heart tissue, which may occur if the surface area of the old epicardial anchor is too small, which can cause a dimpling effect along the surface of the heart, thus reducing the tension of the prosthetic heart valve system as well as increasing the potential for necrosis along the contact area of the heart to the old epicardial anchor device. The new epicardial pad may have a larger surface area to reduce or eliminate the dimpling effect, as the larger surface area may distribute force over a larger area of tissue. This replacement procedure may be technically difficult if the excess length of tether is relatively short, as it may be difficult to grip the tether while also sliding the old epicardial anchor device over the tether, and it may also be difficult to slide a new epicardial anchor device onto the excess tether length without a leader to provide guidance.

According to one aspect of the disclosure, a system comprises:
a prosthetic heart valve having a frame, prosthetic leaflets within the frame configured to allow blood to flow through the prosthetic heart valve in an antegrade direction but to substantially block blood from flowing through the prosthetic heart valve in a retrograde direction;
an anchor configured to be secured against a ventricular apex of a heart of a patient; a tether having a first end, a second end, and an intermediate portion between the first end and the second end, the first end of the tether coupled to the frame, the intermediate portion configured to be received within the anchor, and the second end configured to extend beyond the anchor when the tether is fixed to the anchor; and
a tether adjustment member having a collapsible tube defining an open leading end configured to receive the second end of the tether, and a wire leader extending from a second trailing end of the collapsible tube; and/or
the tube is configured to be secured to the tether via a suture; and/or
the tube includes at least one tooth extending radially inwardly and proximally from the tube, the at least one tooth configured to allow the tube to slide in only one direction over the tether; and/or
the tube is formed from braided wires; and/or
the tube has a large diameter condition and a small diameter condition, wherein the tube has the large diameter condition in the absence of applied forces to the tube, the tube in the large diameter condition having a larger diameter than the tube in the small diameter condition; and/or
in a first configuration, the braid provides a first compressive force to the second end of the tether, and, in a second configuration, the braid provides a second compressive force to the second end of the tether, the second compressive force being greater than the first compressive force.

According to another aspect of the disclosure, a method of adjusting tension on a tether that is coupled to a prosthetic heart valve implanted within a heart of a patient comprises:
accessing the tether, the tether having a first end coupled to the prosthetic heart valve, a second end extending from the anchor, and an intermediate portion received within the anchor, the intermediate portion being between the first end and the second end;
after accessing the tether, engaging the second end of the tether with a collapsible tube so that the second end of the tether is received within a first leading end of the collapsible tube;
securing the collapsible tube to the tether; and
tensioning the tether by applying a proximally directed force to the collapsible tube; and/or
securing the collapsible tube to the tether includes suturing the first leading end of the collapsible tube to the second end of the tether; and/or
securing the collapsible tube to the tether includes engaging the second end of tether with one or more teeth extending radially inwardly from the collapsible tube; and/or
prior to engaging the collapsible tube with the tether, assembling two or more funnel portions, and positioning a narrow end of the assembled funnel portions within the first leading end of the collapsible tube;
wherein engaging the tether with the collapsible tube includes passing the tether through a large funnel end of the assembled funnel portions; and
detaching the two or more funnel portions from the collapsible tube after passing the tether through the large funnel end; and/or
a leader is secured to the collapsible tube, the leader having a braided section and a solid wire section, the method further comprising engaging the braided section with a tensioning instrument prior to tensioning the tether; and/or
engaging the braided section with the tensioning instrument includes one of pinning the braided section or wrapping the braided section around a portion of the tensioning instrument; and/or
tensioning the collapsible tube applies a compressive force to the second end of the tether; and/or
tensioning the collapsible tube includes decreasing a diameter of the braid to apply the compressive force to the second end of the tether; and/or
the anchor is locked to the intermediate portion of the tether, the method further comprising unlocking the anchor from the intermediate portion of the tether; and/or
the anchor is locked to the intermediate portion of the tether through a pinning mechanism and unlocking the anchor from the intermediate portion of the tether includes unlocking the pinning mechanism; and/or
tensioning the collapsible tube includes pulling a leader attached to the collapsible tube; and/or
cutting the tether after tensioning the tether to decouple the collapsible tube from the tether.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A system, comprising:
a prosthetic heart valve having a frame, prosthetic leaflets within the frame configured to allow blood to flow through the prosthetic heart valve in an antegrade direction but to substantially block blood from flowing through the prosthetic heart valve in a retrograde direction;
an anchor configured to be secured against a ventricular apex of a heart of a patient;
a tether having a first end, a second end, and an intermediate portion between the first end and the second end, the first end of the tether coupled to the frame, the intermediate portion configured to be received within the anchor, and the second end configured to extend beyond the anchor when the tether is fixed to the anchor; and
a tether adjustment member having a collapsible tube defining an open leading end configured to receive the second end of the tether, and a wire leader extending from a second trailing end of the collapsible tube.

2. The system of claim 1, wherein the tube is configured to be secured to the tether via a suture.

3. The system of claim 1, wherein the tube includes at least one tooth extending radially inwardly and proximally from the tube, the at least one tooth configured to allow the tube to slide in only one direction over the tether.

4. The system of claim 1, wherein the tube is formed from braided wires.

5. The system of claim 4, wherein the tube has a large diameter condition and a small diameter condition, wherein the tube has the large diameter condition in the absence of applied forces to the tube, the tube in the large diameter condition having a larger diameter than the tube in the small diameter condition.

6. The system of claim 4, wherein, in a first configuration, the braid provides a first compressive force to the second end of the tether, and, in a second configuration, the braid provides a second compressive force to the second end of the tether, the second compressive force being greater than the first compressive force.

7. The system of claim 1, further comprising one or more funnel portions received in the leading end of the collapsible tube.

8. The system of claim 1, wherein the wire leader has a braided section and a solid wire section.

9. A method of adjusting tension on a tether that is coupled to a prosthetic heart valve implanted within a heart of a patient, the method comprising:
accessing the tether, the tether having a first end coupled to the prosthetic heart valve, a second end extending from the anchor, and an intermediate portion received within the anchor, the intermediate portion being between the first end and the second end;
after accessing the tether, engaging the second end of the tether with a collapsible tube so that the second end of the tether is received within a first leading end of the collapsible tube;
securing the collapsible tube to the tether; and
tensioning the tether by applying a proximally directed force to the collapsible tube.

10. The method of claim 9, wherein securing the collapsible tube to the tether includes suturing the first leading end of the collapsible tube to the second end of the tether.

11. The method of claim 9, wherein securing the collapsible tube to the tether includes engaging the second end of tether with one or more teeth extending radially inwardly from the collapsible tube.

12. The method of claim 9, further comprising:
prior to engaging the collapsible tube with the tether, assembling two or more funnel portions, and positioning a narrow end of the assembled funnel portions within the first leading end of the collapsible tube;
wherein engaging the tether with the collapsible tube includes passing the tether through a large funnel end of the assembled funnel portions; and
detaching the two or more funnel portions from the collapsible tube after passing the tether through the large funnel end.

13. The method of claim 9, wherein a leader is secured to the collapsible tube, the leader having a braided section and a solid wire section, the method further comprising engaging the braided section with a tensioning instrument prior to tensioning the tether.

14. The method of claim 13, wherein engaging the braided section with the tensioning instrument includes one of pinning the braided section or wrapping the braided section around a portion of the tensioning instrument.

15. The method of claim 9, wherein tensioning the collapsible tube applies a compressive force to the second end of the tether.

16. The method of claim 15, wherein tensioning the collapsible tube includes decreasing a diameter of the braid to apply the compressive force to the second end of the tether.

17. The method of claim 9, wherein the anchor is locked to the intermediate portion of the tether, the method further comprising unlocking the anchor from the intermediate portion of the tether.

18. The method of claim 17, wherein the anchor is locked to the intermediate portion of the tether through a pinning mechanism and unlocking the anchor from the intermediate portion of the tether includes unlocking the pinning mechanism.

19. The method of claim 9, wherein tensioning the collapsible tube includes pulling a leader attached to the collapsible tube.

20. The method of claim 9, further comprising cutting the tether after tensioning the tether to decouple the collapsible tube from the tether.
